(19) United States
(12) Patent Application Publication
(10) Pub. No.: US 2016/0324744 A1
(43) Pub. Date: Nov. 10, 2016

(54) PROCESS FOR DYEING KERATIN MATERIALS USING POWDER FROM INDIGO-PRODUCING PLANTS AND ALKALINE AGENT(S)

(71) Applicant: L'OREAL, Paris (FR)

(72) Inventors: Patrick CHOISY, Montlouis Sur Loire (FR); Emilie GUENAULT, Saint Roch (FR)

(21) Appl. No.: 15/102,018

(22) PCT Filed: Dec. 2, 2014
(86) PCT No.: PCT/EP2014/076279

§ 371 Date: Jun. 6, 2016

(30) Foreign Application Priority Data
Dec. 6, 2013 (FR) 1362227

(51) Int. Cl. A61K 8/49; A45D 2/00; A61Q 5/06; A61K 8/19; A61K 8/97; A61K 8/22

(52) U.S. Cl. CPC A61K 8/492; A61K 8/97; A61K 8/22; A61Q 5/065; A61K 8/19; A45D 2/001; A61K 2800/4322; A61K 2800/49; A61K 2800/81; A61K 2800/884

(57) ABSTRACT

The invention relates (I) to a process for dyeing keratin materials, in particular keratin fibres, preferably human keratin fibres such as the hair, using i) indigo-producing plant powder, ii) at least one alkaline agent as a post-treatment, preferably an alkali metal or alkaline-earth metal (bi) carbonate and iii) optionally a chemical oxidizing agent such as hydrogen peroxide or a hydrogen peroxide-generating system in co-treatment with i), and it being understood that the composition comprising the alkaline agent(s) is at a pH inclusively between 7.5 and 11.5; and (II) to the use iii) of alkaline agent(s) for fixing the blue colour of and/or for improving the dyeing kinetics of keratin materials dyed with indigo.

The invention relates (I) to a process for dyeing keratin materials, in particular keratin fibres, preferably human keratin fibres such as the hair, using i) indigo-producing plant powder, ii) at least one alkaline agent as a post-treatment, preferably an alkali metal or alkaline-earth metal (bi)carbonate and iii) optionally a chemical oxidizing agent such as hydrogen peroxide or a hydrogen peroxide-generating system in co-treatment with i), and it being understood that the composition comprising the alkaline agent(s) is at a pH inclusively between 7.5 and 11.5; and (II) to the use iii) of alkaline agent(s) for fixing the blue colour of and/or for improving the dyeing kinetics of keratin materials dyed with indigo.

Two major methods for dyeing human keratin fibres, and in particular the hair, are known.

The first, known as oxidation dyeing or permanent dyeing, consists in using one or more oxidation dye precursors, more particularly one or more oxidation bases optionally combined with one or more couplers.

Oxidation bases are usually selected from ortho- or para-phenylenediamines, ortho- or para-aminophenols, and heterocyclic compounds. These oxidation bases are colourless or weakly coloured compounds, which, when combined with oxidizing products, can give rise via a process of oxidative condensation to coloured species, which remain trapped within the fibre.

The shades obtained with these oxidation bases are often varied by combining them with one or more couplers, these couplers being chosen especially from aromatic meta-diamines, meta-aminophenols, meta-diphenols and certain heterocyclic compounds, such as indole compounds.

The variety of molecules used as oxidation bases and couplers allows a wide range of colours to be obtained.

The second dyeing method, known as direct dyeing or semi-permanent dyeing, comprises the application of direct dyes, which are coloured and colouring molecules that have affinity for fibres. Given the nature of the molecules used, they tend rather to remain on the surface of the fibre and penetrate relatively little into the fibre, when compared with the small molecules of oxidation dye precursors. The main advantages of this type of dyeing are that it does not require any oxidizing agent, which limits the degradation of the fibres, and that it does not use any dyes that have particular reactivity, resulting in limitation of the intolerance risks.

The first hair dyes were semi-permanent. One of the most widely-known natural dyes is indigo (see Ullmann's Encyclopedia of Industrial Chemistry, “Hair preparation”, point 5.2.3, 2006 Wiley-VCH Verlag GmbH & Co. KGaA, Weinheim; 10.1002/14356007.a12 571.pub2). Indigo continues to be used in female beauty enhancement for dyeing the hair or the nails, or for dyeing fabrics (jeans), leather, silk, wool, etc. Indigo [482-89-3] is a natural blue dye, and its isomer indirubin is red. Their empirical formula is: C₁₆H₁₀N₂O₂; and their chemical structures are the following:

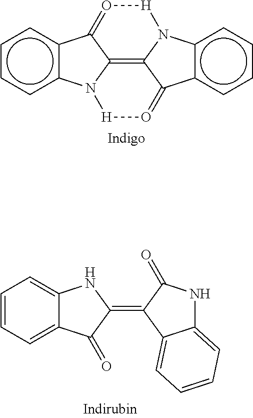

Depending on the oxidation/overoxidation, isatin may give indigotin or indirubin (Maugard et al., 2001). The presence of these two isomers accounts for the violet colour of indigo.

Indigo is derived from indican and may be prepared from various plants known as indigo-producing plants such as Indigofera tinctoria, Indigo suffruticosa, Polygonum tinctorium etc. (see Kirk-Othmer Encyclopedia of Chemical Technology, updated on 17 Apr. 2009. DOI: 10.1002/0471238961.0425051903150618.a01.pub2). The indigo-producing plants are generally chopped and soaked in hot water, heated, fermented and oxidized in the open air to liberate the purple-blue coloured indigo (see Chem. Rev. 2011, 111, 2537-2561, pp. 2537-2561). Indigo is the result of the hydrolysis and then oxidization of indican (glycosyl precursor). The indigo molecule is insoluble in water.

The problem is that dyeing using the indigo leaf is difficult because the kinetics of colour buildup in keratin fibres is very poor. Furthermore, the dyeing process is unstable. Indigo affords a blue colouring on grey hair, and a “cold” colour of ash to violet type on chestnut-brown hair. Nevertheless, the process of dyeing using indigo is difficult to control. In the first minutes, indoxyl is green-yellow, becomes oxidized within hours to indigo and the optimum blue colour is generally reached within a day, and then, beyond 3 to 4 days, the colour “turns” red, which users find undesirable.

To overcome the problem of the poor dyeing kinetics of indigo, it is known practice to “dope” colouring by adding direct dyes that are generally used in direct dyeing, such as nitrobenzene, anthraquinone, nitropyridine, azo, methine, azomethine, xanthene, acridine, azine or triarylmethane direct dyes (see, for example, EP 0 806 199). This option has the drawback for natural product users, or for partisans of “natural/bio” products, in that the colouring is partly performed using synthetic dyes.

Moreover, the colourings obtained with indigo are not always homogeneous between the root and the end or from one fibre to another (The Science of Hair Care, C. Bouillon, J. Wilkinson, 2d Ed., CRC Press, Taylor & Francis Group; Boca Raton, London, pp. 236-241 (2005)).

There is thus a real need to develop dyeing processes which make it possible to obtain powerful, aesthetic colourings within hours of application using indigo, while at the same time respecting the cosmetic nature of the keratin fibres, and which make it possible especially to obtain rapid colourings that are less aggressive to the hair and that, at the same time, withstand external agents (light, bad weather or shampooing), that are persistent and/or homogeneous, while at the same time remaining powerful and/or chromatic, and that do not turn red over time.

This aim is achieved by the present invention, one subject of which is a process for dyeing keratin materials, in particular keratin fibres, preferably human keratin fibres such as the hair, using:
indigo-producing plant powder i) which is mixed before use in an aqueous composition A to obtain a composition B, preferably in the form of a poultice;
a cosmetic alkaline composition C with a pH inclusively between 7.5 and 11.5 and containing:
ii) one or more alkaline agents preferably chosen from carbonates and bicarbonates or mixtures thereof; hydroxides of alkali metals such as sodium or potassium, alkanolamines such as tris(hydroxymethyl)aminomethane;
optionally:
iii) hydrogen peroxide or one or more hydrogen peroxide-generating system(s); and/or
iv) one or more metal salts;
   it being understood that:
composition B and the ingredient(s) iii) and/or iv) are applied to the keratin fibres together with ingredient i), i.e. as a co-treatment, and
composition C is applied to the keratin fibres after the application of composition B, i.e. as a post-treatment.

A subject of the invention is also the use of alkaline agent(s) ii) as defined below and/or of metal salt(s) iv) as defined below, and/or of surfactant(s) v) as defined below, for fixing the blue colour of keratin materials, in particular of keratin fibres such as the hair, dyed with indigo, i.e. limiting the change of the colour towards red, and preferentially the use of the ingredients ii) or iv) and v) for limiting the change of the colour towards red over time and/or for improving the dyeing kinetics of keratin materials, in particular of keratin fibres such as the hair, dyed with indigo, especially in terms of power and intensity of colouring.

The process for dyeing keratin materials according to the invention has the advantage of dyeing the said materials, especially human keratin fibres, with powerful, chromatic dyeing results that are resistant to washing, perspiration, sebum and light, and that are moreover long-lasting, without impairing the said fibres. Furthermore, the colourings obtained using the process give uniform colours from the root to the end of a fibre (little colouring selectivity). The treated keratin fibres have a very pleasant cosmetic appearance and their integrity is respected.

### i) Indigo-Producing Plant Powder

The process of the invention uses, as first ingredient, indigo-producing plant powder.

As indigo-producing plants, mention may be made of numerous species derived from the following genera:
Indigofera such as Indigofera tinctoria, Indigo suffruticosa, Indigofera articulata, Indigofera arrecta, Indigofera gerardiana, Indigofera argenta, Indigofera indica, Indigofera longiracemosa;
Isatis such as Isatis tinctoria;
Polygonum or Persicaria such as Polygonum tinctorium (Persicaria tinctoria);
Wrightia such as Wrightia tinctoria;
Calanthe such as Calanthe veratrifolia; and
Baphicacanthus such as Baphicacanthus cusia.

Preferably, the indigo-producing plant is of the genus Indigofera and more particularly is Indigofera tinctoria.

Use may be made of all or part (in particular the leaves especially for Indigofera tinctoria) of the indigo-producing plant.

The indigo-producing plant powder may be screened to obtain particles with upper limit sizes corresponding to the orifices or mesh sizes of the screen particularly between 35 and 80 mesh (US).

According to a particular mode of the invention, the size of the indigo-producing plant powder particles is fine. According to the invention, a particle size of less than or equal to 500 μm is more particularly intended. Preferentially, the powder consists of fine particles with sizes inclusively between 50 and 300 μm and more particularly between 10 and 200 μm.

It is understood that the said indigo-producing plant particles preferentially have a moisture content of between 0% and 10% by weight relative to the total weight of the powders.

According to a particular embodiment of the invention, the indigo-producing plant powder i) used in the process of the invention is in an aqueous composition A, in an amount inclusively between, in particular, 10% and 99% by weight, relative to the total weight of composition A, more particularly between 10% and 70% by weight, preferentially between 20% and 60% by weight and more preferentially between 25% and 50% by weight.

Composition A, and also compositions B and C as defined previously, are cosmetic compositions, i.e. they are cosmetically acceptable and are therefore suitable for use for application to keratin materials, especially for application to keratin fibres, such as the hair. They are preferably aqueous compositions.

According to a particular embodiment of the invention, the composition A or B of the invention may also contain the ingredients iii) hydrogen peroxide or one or more hydrogen peroxide-generating systems, iv) one or more metal salts as defined below.

Composition B used in the process of the invention is derived from the mixture between the indigo-producing plant powder i) in compact or loose form, and an aqueous composition A as defined previously and preferably water.

Preferably, composition B used in the process of the invention is in the form of a poultice.

According to an advantageous variant, composition B used in the dyeing process also comprises ii) one or more chemical oxidizing agents such as hydrogen peroxide or a hydrogen peroxide-generating system; and iv) optionally one or more metal salts, in particular iron salts.

To do this, i) the indigo-producing plant powder and optionally iii), iv) as defined previously and/or v) one or more surfactants, preferably anionic or nonionic surfactants, is (are) mixed with an aqueous composition A, and preferentially mixed with water, to obtain a poultice B of creamy and pleasant consistency. When the indigo-producing plant powder is compact, it is crumbled into the aqueous composition A, and the compact composition is preferentially crumbled into water. The ratios of indigo-producing plant powder i) according to the invention and of the aqueous composition A and preferentially water for obtaining the composition B in the form of a poultice preferably range from 1 part by weight of i) per 1 part by weight of aqueous composition A and preferentially of water, of oil and of other cosmetic adjuvants (1/1) to 1 part by weight of i) per 4 parts by weight of aqueous composition and preferentially of water (1/4), of aqueous composition A and preferentially of water, of oil or of cosmetic emulsion (1/3).

According to another particular embodiment of the invention, composition B comprises, besides the ingredients ii) to v) as defined previously, other ingredients of natural origin.

During the preparation of the poultice, one or more identical or different clays may be added.

### The Organic Solvents:

Compositions A, B and/or C as defined previously may comprise one or more organic solvents. Examples of preferred organic solvents include C₁-C₄ lower alkanols, such as ethanol and isopropanol; polyols and polyol ethers such as 2-butoxyethanol, propylene glycol, propylene glycol monomethyl ether, diethylene glycol monoethyl ether and monomethyl ether, hexylene glycol, and also aromatic alcohols, for instance benzyl alcohol or phenoxyethanol.

The organic solvents are present in proportions preferably of between 0.1% and 20% by weight approximately and even more preferentially between 0.5% and 10% by weight approximately relative to the total weight of the composition under consideration.

Compositions A, B and/or C as defined previously may comprise one or more identical or different oils.

The term “oil” means a “fatty substance” that is liquid at room temperature (25° C.) and at atmospheric pressure (760 mmHg); the viscosity at 25° C. is preferably less than 1200 cps and better still less than 500 cps (defined, for example, from the Newtonian plateau determined using an ARG2 rheometer from TA Instruments equipped with a spindle with cone-plate geometry 60 mm in diameter and with an angle of 2 degrees over a shear stress range of from 0.1 Pa to 100 Pa).

The term “fatty substance” means an organic compound that is insoluble in water at ordinary temperature (25° C.) and at atmospheric pressure (760 mmHg) (solubility of less than 5%, preferably less than 1% and even more preferentially less than 0.1%). They bear in their structure at least one hydrocarbon-based chain comprising at least 6 carbon atoms or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

The term “non-silicone oil” means an oil not containing any silicon atoms (Si) and the term “silicone oil” means an oil containing at least one silicon atom.

More particularly, the oils are chosen from non-silicone oils and in particular C₆-C₁₆ hydrocarbons or hydrocarbons containing more than 16 carbon atoms and in particular alkanes; oils of animal origin; triglyceride oils of plant origin; essential oils; fluoro oils or glycerides of synthetic origin, fatty alcohols; fatty acid and/or fatty alcohol esters other than triglycerides, fatty acid amides and silicone oils.

Preferably, the oils are not oxyalkylenated or glycerolated ethers.

Preferably, the oils do not comprise any C₂-C₃ oxyalkylene units or any glycerolated units.

Preferably, the oils are not fatty acids which, in salified form, give water-soluble soaps.

The oils that may be used as second ingredient b) in composition A or B in accordance with the invention may be silicones.

The silicones may be volatile or non-volatile, cyclic, linear or branched silicones, which are unmodified or modified with organic groups, having a viscosity from 5×10⁻⁶ to 2.5 m²/s at 25° C., and preferably 1×10⁻⁵ to 1 m²/s.

Preferably, the silicone is chosen from polydialkylsiloxanes, in particular polydimethylsiloxanes (PDMSs), and organomodified polysiloxanes comprising at least one functional group chosen from poly(oxyalkylene) groups, amino groups and alkoxy groups.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably from 4 to 5 silicon atoms. These are, for example, octamethylcyclotetrasiloxane sold especially under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.
   Mention may also be made of cyclocopolymers of the dimethylsiloxane/methylalkylsiloxane type, such as Volatile Silicone® FZ 3109 sold by the company Union Carbide, of formula:

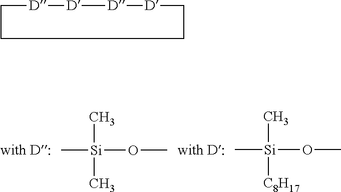

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(hexa-2,2,2′,2′,3,3′-trimethylsilyloxy)neopentane;
(ii) linear volatile polydialkylsiloxanes containing 2 to 9 silicon atoms and having a viscosity of less than or equal to 5×10⁻⁶ m²/s at 25° C. An example is decamethyltetrasiloxane sold especially under the name SH 200 by the company Toray Silicone. Silicones belonging to this category are also described in the article published in Cosmetics and Toiletries. Vol. 91, January 76, pp. 27-32, Todd & Byers, Volatile Silicone Fluids for Cosmetics.
   Use is preferably made of non-volatile polydialkylsiloxanes, polydialkylsiloxane gums and resins, polyorganosiloxanes modified with the organofunctional groups above, and mixtures thereof.
   These silicones are more particularly chosen from polydialkylsiloxanes, among which mention may be made mainly of polydimethylsiloxanes bearing trimethylsilyl end groups. The viscosity of the silicones is measured at 25° C. according to Standard ASTM 445 Appendix C.

Among these polydialkylsiloxanes, mention may be made, in a nonlimiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups known under the name Dimethiconol (CTFA), such as the oils of the 48 series from the company Rhodia.

Compositions A, B and/or C as defined previously may comprise one or more oils, chosen from fatty alcohols, fatty acid amides and fatty acid esters in the form of oils.

It is recalled that, for the purposes of the invention, fatty alcohols, esters and acids more particularly have at least one linear or branched, saturated or unsaturated hydrocarbon-based group comprising 6 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

More precisely, these compounds may represent an ester of a C₁-C₁₀ alcohol and of a C₆-C₃₀ fatty acid such as R—C(O)—O—R′ with R representing a linear or branched C₆-C₃₀ alkyl or linear or branched C₆-C₃₀ alkenyl group, comprising one or two unsaturations, and R representing a linear or branched C₁-C₁₀ alkyl group.

Preferentially. R represents a linear C₁₀-C₂₀ alkyl group and R′ represents a C₁-C₆ alkyl group that is preferably branched, such as isopropyl myristate.

According to another advantageous variant, the ingredient ii) represents one or more amides of a C₆-C₃₀ fatty acid and of a primary or secondary, preferably primary. C₁-C₁₀ amine, such as those of formula R″—C(O)—N(Rₐ)—R′″ with R″ representing a linear or branched C₆-C₃₀ alkyl or a linear or branched C₆-C₃₀ alkenyl group, comprising one or two unsaturations, which may be substituted with one or more hydroxyl groups, or (di)(C₁-C₆)(alkyl)amino, and R′″ representing a linear or branched C₁-C₁₀ alkyl group. Rₐ representing a hydrogen atom or an alkyl group as defined for R′″. Preferably, R″ represents a C₁₄-C₂₀ alkenyl group, Rₐ represents a hydrogen atom and R′″ represents a C₁-C₆ alkyl group optionally substituted with (di)(C₁-C₄)(alkyl)amino such as oleylamidopropyldimethylannine.

As regards the C₆-C₁₆ alkanes, they are linear or branched, and possibly cyclic. Examples that may be mentioned include hexane, dodecane and isoparaffins such as isohexadecane and isodecane. The linear or branched hydrocarbons containing more than 16 carbon atoms may be chosen from liquid paraffins, petroleum jelly, liquid petroleum jelly, polydecenes, and hydrogenated polyisobutene such as Parleam®.

Among the animal oils, mention may be made of perhydrosqualene.

Among the triglycerides of plant or synthetic origin, mention may be made of liquid fatty acid triglycerides containing from 6 to 30 carbon atoms, for instance heptanoic or octanoic acid triglycerides, or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides, for instance those sold by the company Stéarineries Dubois or those sold under the names Miglyol® 810, 812 and 818 by the company Dynamit Nobel, jojoba oil and shea butter oil.

Among the fluoro oils, mention may be made of perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-(trifluoromethyl)perfluoromorpholine sold under the name PF 5052® by the company 3M.

Among the essential oils contained in the composition of the invention, mention may be made of those mentioned in Ullmann's Encyclopedia of Industrial Chemistry (“Flavors and Fragrances”, Karl-Georg Fahlbusch et al., Published Online: 15 Jan. 2003, DOI: 10.1002/14356007.a11_141).

Preferably, the oil(s) of the invention are non-silicone oils. The term “non-silicone oil” is intended to mean an oil not containing any silicon atoms (Si) and the term “silicone oil” is intended to mean an oil containing at least one silicon atom.

According to a preferred variant of the invention, the oil(s) are chosen from C₆-C₁₆ alkanes, polydecenes, liquid esters of a fatty acid and/or of a fatty alcohol, and liquid fatty alcohols, or mixtures thereof.

Better still, the oils are chosen from liquid petroleum jelly. C₆-C₁₆ alkanes and polydecenes.

In this preferred variant, the oil(s) are chosen from mineral oils such as liquid petroleum jelly.

According to another most particularly preferred mode of the invention, the oils are chosen from oils of natural origin, more particularly oils of plant origin, preferentially chosen from jojoba oil, babassu oil, sunflower oil, olive oil, coconut oil, Brazil nut oil, marula oil, corn oil, argan oil, soybean oil, marrow oil, grapeseed oil, linseed oil, sesame oil, hazelnut oil, apricot oil, macadamia oil, arara oil, coriander oil, almond oil, castor oil, avocado oil, shea butter oil, rapeseed oil, borage oil, evening primrose oil, pomegranate oil, mango oil, palm oil, cottonseed oil and copra oil.

More particularly, compositions A, B and/or C contain one or more oils of plant origin preferably chosen from avocado oil, olive oil, coconut oil, copra oil, argan oil and sunflower oil; more preferentially, the oil(s) of the invention are chosen from copra oils.

Compositions A, B and/or C used in the process of the invention preferably comprise one or more oils in an amount inclusively between 1% and 80% by weight, more particularly between 2% and 50% by weight, preferentially between 3% and 40% by weight and more preferentially between 5% and 25% by weight, relative to the total weight of the said compositions.

### The Adjuvants:

Compositions A, B and C used in the process of the invention may also contain various adjuvants conventionally used in hair dye compositions, such as mineral or organic thickeners, and in particular anionic, cationic, nonionic and amphoteric polymeric associative thickeners, antioxidants, penetrants, sequestrants, fragrances, buffers, dispersants, conditioning agents, for instance ceramides, film-forming agents, preserving agents, opacifiers and mineral or organic thickeners such as clays.

The above adjuvants are generally present in an amount for each of them of between 0.01% and 40% by weight relative to the weight of the composition, and preferably between 0.1% and 20% by weight relative to the weight of the composition under consideration.

Needless to say, a person skilled in the art will take care to select this or these optional additional compound(s) such that the advantageous properties intrinsically associated with the composition or the poultice that are useful in the dyeing process in accordance with the invention are not, or are not substantially, adversely affected by the envisaged addition(s).

### The Additional Dyes:

Compositions A or B used in the dyeing process of the invention may also contain one or more additional direct dyes other than the indigo-producing plant powder i).

These direct dyes are chosen, for example, from those conventionally used in direct dyeing, and among which mention may be made of any commonly used aromatic and/or non-aromatic dyes such as neutral, acidic or cationic nitrobenzene direct dyes, neutral, acidic or cationic azo direct dyes, natural direct dyes, neutral, acidic or cationic quinone and in particular anthraquinone direct dyes, azine, triarylmethane, indoamine, methine, styryl, porphyrin, metalloporphyrin, phthalocyanine, methine cyanine direct dyes, and fluorescent dyes.

Preferentially, compositions A or B used in the process of the invention comprise one or more natural dyes other than indigo i) as defined previously. Among the natural direct dyes, mention may be made of condensed, gallic or ellagic tannins, naphthoquinones (juglone, lawsone), anthraquinones (emodin, alizarin, etc.), isatin, curcumin, spinulosin, polyphenols such as flavonoids, isoflavonoids, pterocarpans, neoflavones or orceins. These natural dyes may be added in the form of defined compounds, extracts or plant parts. The said defined compounds from extracts or from plant parts are preferably in the form of powders, in particular fine powders whose particles have sizes identical to that of the indigo-producing plant powder as defined previously.

The natural or non-natural direct dye(s), other than the indigo-producing plant powder i), used in the process of the invention particularly represents from 0.001% to 10% by weight relative to the total weight of the composition and even more preferentially from 0.05% to 5% by weight relative to the total weight of the composition under consideration.

Preferably, the composition of the invention does not contain any synthetic direct dyes, i.e. dyes that do not occur in nature.

Compositions A or B used in the process of the invention may also comprise one or more oxidation bases and/or one or more couplers conventionally used for dyeing keratin fibres.

Among the oxidation bases, mention may be made of para-phenylenediamines, bis(phenyl)alkylenediamines, para-aminophenols, bis-para-aminophenols, ortho-aminophenols and heterocyclic bases, and the addition salts thereof.

Among these couplers, mention may be made especially of meta-phenylenediamines, meta-aminophenols, meta-diphenols, naphthalene-based couplers and heterocyclic couplers, and the addition salts thereof.

The oxidation base(s) present in the composition(s) are each generally present in an amount of between 0.001% and 10% by weight, relative to the total weight of the dye composition(s).

Preferably, the dyeing process of the invention does not use any oxidation dyes.

### pH of Compositions A and B

According to a particular mode of the invention, the pH of the aqueous composition A containing the ingredients i), and also the pH of the aqueous composition B, is neutral, i.e. the pH is about 5 (preferably ranging from 3 to 8 and better still from 4.5 to 7.5).

According to a particular mode of the invention, composition A and/or B is acidic and preferably has a pH ranging from 2 to 6.5, particularly ranging from 3 to 6, and preferentially the pH=5.

The pH of composition A and/or B may be adjusted to the desired value by means of acidic or alkaline agents usually used in the dyeing of keratin fibres or alternatively with the aid of standard buffer systems, or of clays as defined previously present in composition B or in the aqueous composition A.

Among the acidic agents that may be used in the compositions of the invention, mention may be made of mineral or organic acids, for instance hydrochloric acid, orthophosphoric acid or sulfuric acid, carboxylic acids, for instance acetic acid, tartaric acid, citric acid and lactic acid, and sulfonic acids; the acid is preferably an organic acid such as citric acid.

Among the alkaline agents, mention may be made of the agents ii) below.

### ii) The Alkaline Agents and Composition C

According to a particular embodiment of the invention, the dyeing process uses ii) one or more alkaline agents applied as a post-treatment, i.e. after the application of ingredient i) and optionally the ingredient(s) iii) and/or iv).

Preferably, the dyeing process of the invention uses one or more organic or mineral, preferably mineral, alkaline agents.

The alkaline agent(s) are preferably chosen from those with a pK_{b} at 25° C. of less than 12, preferably less than 10 and even more advantageously less than 6. It should be noted that it is the pK_{b} corresponding to the function of highest basicity.

More particularly, the alkaline agent(s) ii) are chosen from aqueous ammonia, alkali metal or alkaline-earth metal hydroxides, preferably of sodium or potassium, buffered with one or more amino acids such as glycine, carbonates, bicarbonates or hydrogen carbonates, carbonates buffered with bicarbonate to obtain a pH<12.0, phosphates of alkali metals or of alkaline-earth metals such as sodium or potassium, alkanolamines such as mono-, di- and triethanolamine, mono-, di- and tri(hydroxymethyl)aminomethane, and also derivatives thereof, amino acids, and the compounds of formula (I) below:

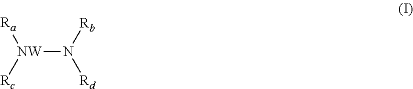

in which formula (I) W is a (C₁-C₆)alkylene group such as propylene, optionally substituted with a hydroxyl or amino group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

Examples of amines of formula (I) that may be mentioned include 1,3-diaminopropane, 1,3-diamino-2-propanol, spermine and spermidine.

The term “alkanolamine” means an organic amine comprising a primary, secondary or tertiary amine function, and one or more linear or branched C₁-C₈ alkyl groups bearing one or more hydroxyl radicals.

Organic amines chosen from alkanolamines such as monoalkanolamines, dialkanolamines or trialkanolamines comprising one to three identical or different C₁-C₄ hydroxyalkyl radicals are in particular suitable for performing the invention.

Among the compounds of this type, mention may be made of monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethyl)aminomethane.

More particularly, the amino acids that can be used are of natural or synthetic origin, in their L. D or racemic form, and comprise at least one acid function chosen more particularly from carboxylic acid, sulfonic acid, phosphonic acid and phosphoric acid functions. The amino acids may be in neutral or ionic form.

As amino acids that may be used in the present invention, mention may be made especially of aspartic acid, glutamic acid, alanine, arginine, ornithine, citrulline, asparagine, carnitine, cysteine, glutamine, glycine, histidine, lysine, isoleucine, leucine, methionine. N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (II) below, and also the salts thereof:

R—CH₂—CH(NH₂)—C(O)—OH (II)

in which formula (II) R represents a group chosen from: imidazolyl, preferably 5-imidazolyl; —(CH₂)₃—NH₂; —(CH₂)₂—NH₂; —(CH₂)₂N(H)—C(O)—NH₂; and —(CH₂)₂—N(H)—C(NH)—NH₂.

The compounds corresponding to formula (II) are histidine, lysine, arginine, ornithine and citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that can be used in the present invention, mention may be made in particular of carnosine, anserine and balenine.

The organic amine may also be chosen from compounds comprising a guanidine function. As amines of this type that may be used in the present invention, besides arginine, which has already been mentioned as an amino acid, mention may be made especially of creatine, creatinine, 1,1-dimethylguanidine, 1,1-diethylguanidine, glycocyamine, metformin, agmatine. N-amidinoalanine, 3-guanidinopropionic acid, 4-guanidinobutyric acid and 2-([amino(imino)methyl]amino)ethane-1-sulfonic acid.

Hybrid compounds that may be mentioned include the salts of the amines mentioned previously with acids such as carbonic acid or hydrochloric acid.

According to a particularly advantageous mode of the invention, the dyeing process uses iii) one or more (bi)carbonates.

The term “(bi)carbonates” is understood to mean:
a) carbonates of alkali metals (Met₂⁺, CO₃²⁻), of alkaline-earth metals (Met²⁺, CO₃²⁻), of ammonium ((R″₄N⁺)₂, CO₃²⁻) or of phosphonium ((R″₄P⁺)₂, CO₃²⁻) with Met′ representing an alkaline-earth metal and Met representing an alkali metal, and R″, which may be identical or different, represent a hydrogen atom, an optionally substituted (C₁-C₆)alkyl group such as a hydroxyethyl group), and
b) bicarbonates, also known as hydrogen carbonates, of the following formulae:
   R′⁺, HCO₃⁻ with R′ representing a hydrogen atom, an alkali metal, an ammonium group R″₄N⁺— or a phosphonium group R″₄P⁺— in which R″, which may be identical or different, represent a hydrogen atom, an optionally substituted (C₁-C₆)alkyl group such as hydroxyethyl and, when R′ represents a hydrogen atom, the hydrogen carbonate is then known as a dihydrogen carbonate (CO₂, H₂O); and
   Met′²⁺(HCO₃⁻)₂, with Met′ representing an alkaline-earth metal.

Mention may be made of Na, K, Mg and Ca carbonates or hydrogen carbonates and mixtures thereof, and in particular sodium hydrogen carbonate. These hydrogen carbonates may originate from a natural water, for example spring water from the Vichy basin or from La Roche-Posay or Badoit water (cf. patent, for example the document FR 2 814 943). In particular, mention may be made of sodium carbonate [497-19-8]=Na₂CO₃, sodium hydrogen carbonate or sodium bicarbonate [144-55-8]=NaHCO₃, and sodium dihydrogen carbonate=Na(HCO₃)₂.

More particularly, ii) is chosen from alkali metal or ammonium carbonates, alkali metal or ammonium bicarbonates and also alkali metal carbonates buffered with alkali metal bicarbonates to obtain a pH<12.0, preferentially less than or equal to 11.0 and more preferentially inclusively between 8.0 and 11.0.

According to another particular embodiment of the invention, the alkaline agent(s) ii) of the invention are chosen from hydroxides of alkali metals or of alkaline-earth metals, preferably of sodium or potassium such as NaOH buffered with one or more amino acids such as glycine, to obtain a pH<11.5, preferentially less than or equal to 11.0, more preferentially inclusively between 8.0 and 11.0 and more particularly inclusively between 9.0 and 10.0.

According to another particular embodiment of the invention, the alkaline agent(s) ii) of the invention are chosen from phosphates of alkali metals or of alkaline-earth metals such as of sodium or potassium, and alkanolamines such as tri(hydroxymethyl)aminomethane.

More preferentially, the alkaline agent(s) ii) in composition C as defined previously are chosen from: NaOH/glycine buffer, 0.5 M at pH 10; NaOH/glycine buffer, 0.5 M at pH 9; sodium carbonate/sodium bicarbonate buffer, 0.5 M at pH 11; sodium carbonate/sodium bicarbonate buffer, 0.5 M at pH 10; sodium carbonate/sodium bicarbonate buffer, 0.5 M at pH 9; 0.5 M ammonium bicarbonate, pH 8.5; 0.5 M potassium bicarbonate, pH 8.4; 0.5 M sodium bicarbonate, pH 8.2 and potassium carbonate/potassium bicarbonate buffer, 0.5 M at pH 9.

Preferentially, the alkaline agent(s) are in an aqueous cosmetic composition C as defined previously.

According to the invention, the alkaline agent(s) used preferably represent from 0.001% to 10% by weight relative to the total weight of the composition(s) containing the alkaline agent(s), and even more preferentially from 0.005% to 5% by weight.

### pH of Composition C Used as Post-Treatment

According to a particular mode of the invention, the pH of the aqueous composition C containing the ingredients iii) is basic, i.e. greater than 7.5 and preferably less than or equal to 11.0.

According to a particular mode of the invention, composition C of the invention has a pH ranging from 8.0 to 10.0 and more preferentially ranging from 8.5 to 9.5.

According to the invention, the alkaline agent(s) used are in the aqueous composition C at a concentration inclusively between 0.1 M and 1 M, and preferably at 0.5 M.

iii) Hydrogen Peroxide or Hydrogen Peroxide-Generating System(s)

According to a particular embodiment of the invention, the dyeing process uses iii) hydrogen peroxide or one or more hydrogen peroxide-generating systems. The latter are applied as a co-treatment with ingredient i) as defined previously.

Preferably, the hydrogen peroxide-generating system(s) are chosen from:
a) urea peroxide;
b) polymeric complexes that can release hydrogen peroxide, such as polyvinylpyrrolidone/H₂O₂ in particular in the form of powders, and the other polymeric complexes described in U.S. Pat. No. 5,008,093; U.S. Pat. No. 3,376,110; U.S. Pat. No. 5,183,901;
c) oxidases that produce hydrogen peroxide in the presence of a suitable substrate (for example glucose in the case of glucose oxidase or uric acid with uricase);
d) metal peroxides that generate hydrogen peroxide in water, for instance calcium peroxide or magnesium peroxide;
e) perborates; and
f) percarbonates.

According to a preferred embodiment of the invention, the dyeing process uses iii) one or more hydrogen peroxide-generating systems chosen from a) urea peroxide, b) polymeric complexes that can release hydrogen peroxide, chosen from polyvinylpyrrolidone/H₂O₂; c) oxidases; e) perborates and f) percarbonates.

In particular, iii) represents hydrogen peroxide.

According to a particular mode of the invention, the process uses ingredient iii) which is in a composition.

The composition(s) comprising hydrogen peroxide or the hydrogen peroxide-generating system(s) may also contain various adjuvants or oils conventionally used in hair dye compositions as defined previously.

According to a particular mode of the invention, the hydrogen peroxide or the hydrogen peroxide-generating system(s) used preferably represent from 0.001% to 12% by weight, expressed as hydrogen peroxide, relative to the total weight of the composition(s) containing them, and even more preferentially from 0.2% to 3% by weight, such as 1%.

### iv) The Metal Salts

According to a particular embodiment of the invention, the process of the invention uses iv) one or more metal salts.

According to a particular embodiment of the invention, the metal salt(s) of the invention iv) are used in the dyeing process of the invention as a post-treatment, i.e. after the application of ingredient i).

According to another particular embodiment of the invention, the metal salt(s) of the invention iv) are used in the dyeing process of the invention as a co-treatment, i.e. iv) is applied at the same time as ingredient i), or alternatively they are in the aqueous composition A or else with ingredient i) as defined previously. Preferably, the metal salt(s) are in a poultice B as defined previously.

The metal salt(s) comprise in their structure one or more metals in which the metal atom bears at least one positive or negative charge, preferably a positive charge, and for which, preferentially, the oxidation state is I or II and more preferentially II. In particular, the metal salt(s) of the invention are derived from the action of at least one mineral or organic acid on a metal. Thus, the metal salt(s) of the invention may be organic or mineral.

According to one variant, the metal salt(s) are mineral and are preferentially chosen from hydrated or anhydrous halides, carbonates, sulfates and phosphates, especially sulfates.

According to an advantageous variant, the metal salt(s) comprise as metal a metal chosen from transition metals and rare-earth metals, preferably transition metals. Among the transition metals, mention may be made especially of manganese, iron, zinc, titanium, zirconium, molybdenum, tungsten and vanadium, and among these most particularly of iron.

According to another preferred variant, the metal salts are in oxidation state I or II, preferably II and bear two (poly)hydroxy acid-based ligands.

The term “(poly)hydroxy acid” means any carboxylic acid which comprises a hydrocarbon-based chain which is linear or branched, and saturated or unsaturated, preferably saturated and/or linear, comprising from 1 to 10 carbon atoms and from 0 to 9 hydroxyl groups, preferably 1 to 4 OH groups, and comprising from 1 to 4 carboxylic groups —C(O)—OH, at least one of the said —C(O)—OH functions of which is in the carboxylate form —C(O)—O⁻ complexed with the metal atom, preferably Fe(II).

According to a preferred variant, the metal salt(s) of the invention are organic, and preferentially comprise a metal that is complexed with two carboxylate groups corresponding to formula (III):

R—C(O)—O-M-O—C(O)—R′ (III)

and also the solvates thereof, such as hydrates, and enantiomers thereof;
in which formula (III):
M represents a metal (II) or metal²⁺ in oxidation state II,
R and R′, which may be identical or different, represent a (C₁-C₆)(poly)hydroxyalkyl group.
   In particular, the metal salt(s) are chosen from the organic acid salts of transition metals, especially of iron.

The organic metal salts may be chosen more particularly from organic acid salts such as citrates, lactates, glycolates, gluconates, acetates, propionates, fumarates, oxalates and tartrates, especially acetates, citrates and gluconates; especially gluconates.

According to another particular embodiment, the process for dyeing keratin materials does not use any metal salt(s) or mordant(s) iv).

According to a particular mode of the invention, the metal salt(s) used preferably represent from 0.001% to 12% by weight relative to the total weight of the composition(s) containing them, and even more preferentially from 0.2% to 3% by weight, such as 1%.

### v) The Surfactant(s)

According to a particular embodiment, the dyeing process uses v) one or more surfactants.

Preferably, the surfactant(s) are chosen from anionic and nonionic surfactants.

According to an advantageous variant of the invention, the dyeing process uses one or more nonionic surfactants v).

The term “nonionic surfactant” means an amphiphilic compound that is not capable of dissociating into ions in aqueous solution.

More particularly, the dyeing process uses one or more nonionic surfactants chosen from:
alkoxylated and especially ethoxylated derivatives of i) alcohols, ii) alkylphenols, iii) fatty acids, iv) monoalkanolamides, v) sorbitan esters (Spans and Tweens), vi) alkoxylated fatty acid amines and vii) ethylene oxide-propylene oxide copolymers (occasionally referenced as polymeric surfactants);
polyhydroxylated surfactants such as glycol esters,
surfactants derived from monosaccharides and polysaccharides,
glycerol (and polyglycerol) esters,
glucoside (and polyglucoside) and sucrose esters,
amine oxide, sulfinyl, sulfoxide and phosphine surfactants.

The alkoxylated surfactants may originate from the products of condensation of hydrophobic compounds such as alcohols, phenols, mercaptans, amines, carboxylic acids or carbonamides with oligoglycol ethers, fatty acid esters of (di)glycerol, of sugars, of hydrogenated sugars such as sorbitol, or alkyl(poly)glucosides.

According to a particular mode of the invention, ingredient v) is chosen from alkoxylated and particularly ethoxylated or glycerolated nonionic surfactants, or mixtures thereof.

More particularly, the nonionic surfactant is chosen from:
oxyalkylenated or glycerolated fatty alcohols;
oxyalkylenated alkylphenols, the alkyl chain of which is a C₈-C₁₈ alkyl chain;
oxyalkylenated or glycerolated fatty amides;
oxyalkylenated vegetable oils;
optionally oxyalkylenated C₆-C₃₀ acid esters of sorbitan;
optionally oxyalkylenated fatty acid esters of sucrose;
polyethylene glycol esters of fatty acids;
(C₆-C₃₀)alkylpolyglycosides;
N—(C₆-C₃₀)alkylglucamine derivatives;
amine oxides such as (C₁₀-C₁₄)alkylamine oxides or N-acylaminopropylmorpholine oxides;
copolymers of ethylene oxide and of propylene oxide;
mixtures thereof.

More particularly, the mean number of oxyalkylene units is advantageously between 2 and 150 units. Preferably, they are oxyethylene or oxypropylene units or mixtures thereof.

As regards the glycerolated surfactants, they preferably comprise, on average, from 1 to 20 glycerol groups and in particular from 1.5 to 5.

In accordance with one particularly advantageous embodiment of the invention, the composition comprises at least one nonionic surfactant chosen from oxyalkylenated or glycerolated C₆-C₃₀ alcohols.

According to a particularly advantageous embodiment of the invention, the process for dyeing keratin materials uses one or more nonionic surfactants chosen from sorbitan esters v) (Spans and Tweens), in particular optionally oxyalkylenated and preferably oxyethylenated C₆-C₃₀ acid esters of sorbitan. More particularly, the process for dyeing keratin materials uses one or more nonionic surfactants chosen from C₈-C₃₀ and preferably C₈-C₂₀ fatty acid esters of sorbitan.

Preferably, the surfactant(s) used in the process of the invention are chosen from the compounds of formula (IV) below:

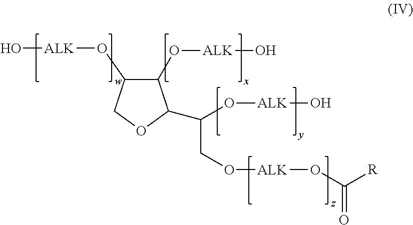

and also optical isomers thereof and hydrates thereof,
in which formula (IV):
ALK, which may be identical or different, preferably identical, represent a linear or branched (C₁-C₆)alkylene group, such as ethylene;
x, y and z, which may be identical or different, represent an integer between 0 and 100 inclusive, preferably between 0 and 50 and more particularly between 0 and 20;
   it being understood that the sum x+y+z is an integer between 1 and 100 inclusive, preferably between 10 and 50 and more particularly between 15 and 30, such as 20;
R represents a linear or branched (C₆-C₃₀)alkyl, preferably (C₈-C₃₀) alkyl and more particularly (C₈-C₂₀)alkyl group.

Use is preferably made of sorbitan monoesters oxyethylenated with a number of moles of ethylene oxide of the ester inclusively between 2 (2 OE) and 40 (OE), particularly between 4 (4 OE) and 20 (20 OE).

The preferred sorbitan esters are oxyethylenated (4 OE) sorbitan monolaurate or polysorbate 21 with w+x+y+z=4 (Tween 21), oxyethylenated sorbitan monolaurate or polysorbate 20 with w+x+y+z=20 (Tween 20), oxyethylenated (4 OE) sorbitan monostearate or polysorbate 61 with w+x+y+z=4 (Tween 61), oxyethylenated (20 OE) sorbitan monostearate or polysorbate 60 with w+x+y+z=20 (Tween 60 and 65); oxyethylenated (50 OE) sorbitan monooleate or polysorbate 81 (Tween 81); oxyethylenated (20 OE) sorbitan monooleate or polysorbate 80 with w+x+y+z=20 (Tween 80).

According to a particular embodiment of the invention, the dyeing process uses one or more nonionic surfactants chosen from sorbitan esters oxyethylenated with a number of moles of ethylene oxide of the ester of between 15 and 30, such as 20 (20 OE).

According to another particular mode of the invention, the process for dyeing keratin materials uses one or more anionic surfactants.

The term “anionic surfactant” means an amphiphilic compound in which the hydrophobic part bears an anionic hydrophilic group with a cationic counterion that is generally metallic, preferably an alkali metal such as Na or K, or ammonium; the hydrophilic group is thus polar and capable of dissociating into an anion in aqueous solution.

The preferred anionic surfactants are surfactants with carboxylate, sulfate, sulfonate, sulfoacetate, sulfosuccinate, phosphate, isethionate, sarcosinate, glutamate, lactylate, taurate, fatty acid salt, galactosideuronic salt or carboxylic ether acid salt anionic groups, and mixtures thereof, more preferentially sulfates such as alkyl sulfates.

More particularly, the anionic surfactant(s) used in the process of the invention are chosen from:
(C₆-C₃₀)alkyl sulfates, (C₆-C₃₀)alkyl ether sulfates, (C₆-C₃₀)alkylamido ether sulfates, alkylaryl polyether sulfates, monoglyceride sulfates;
(C₆-C₃₀)alkyl sulfonates, (C₆-C₃₀)alkylamide sulfonates, (C₆-C₃₀)alkylaryl sulfonates, α-olefin sulfonates, paraffin sulfonates;
(C₆-C₃₀)alkyl phosphates;
(C₆-C₃₀)alkyl sulfosuccinates, (C₆-C₃₀)alkyl ether sulfosuccinates, (C₆-C₃₀)alkylamide sulfosuccinates;
(C₆-C₃₀)alkyl sulfoacetates;
(C₆-C₂₄)acyl sarcosinates;
(C₆-C₂₄)acyl glutamates;
(C₆-C₃₀)alkyl polyglycoside carboxylic ethers; (C₆-C₃₀)alkyl polyglycoside sulfosuccinates;
(C₆-C₃₀)alkyl sulfosuccinamates;
(C₆-C₂₄)acyl isethionates;
N—(C₆-C₂₄)acyl taurates;
fatty acid salts;
(C₈-C₂₀)acyl lactylates;
(C₆-C₃₀)alkyl-D-galactoside uronic acid salts;
polyoxyalkylenated (C₆-C₃₀)alkyl ether carboxylic acid salts, polyoxyalkylenated (C₆-C₃₀)alkylaryl ether carboxylic acid salts, polyoxyalkylenated (C₆-C₃₀)alkylamido ether carboxylic acid salts;
and mixtures thereof;
more preferentially (C₆-C₃₀)alkyl sulfates, (C₆-C₃₀)alkyl ether sulfates, (C₆-C₃₀)alkylamido ether sulfates, alkylaryl polyether sulfates and monoglyceride sulfates.

These anionic surfactants are advantageously in the form of salts in the composition according to the invention, especially of salts of alkali metals, such as sodium; of alkaline-earth metals, for instance magnesium; ammonium salts; amine salts; amino alcohol salts. Depending on the conditions, they may also be in their acid form.

It should be noted that the alkyl or acyl radicals of these various compounds preferably comprise from 12 to 20 carbon atoms. Preferably, the aryl radical denotes a phenyl or benzyl group.

Furthermore, the polyoxyalkylenated anionic surfactants preferably comprise from 2 to 50 alkylene oxide and in particular ethylene oxide groups.

In accordance with a preferred embodiment of the invention, the anionic surfactant(s) are chosen from (C₆-C₃₀)alkyl sulfates such as an alkali metal or alkaline-earth metal dodecyl sulfate, in particular sodium dodecyl sulfate (SDS). This surfactant is sold, for example, by the company Sigma.

According to a particularly advantageous embodiment, the dyeing process of the invention uses one or more surfactants as a co-treatment with i), preferably in composition A or B as defined previously, particularly in the form of a poultice.

According to another advantageous embodiment, the dyeing process of the invention uses one or more surfactants as a post-treatment with ii) or after the application of composition C; preferably, the surfactant(s) v) are in the composition C as defined previously.

According to a preferred embodiment of the invention, the surfactant(s) v) are in composition A, B and/or C as defined previously in an amount of between 0.001% and 20%, particularly between 0.01% and 10% and more particularly between 0.01% and 1%, such as 0.1% by weight, relative to the total weight of composition B and/or C.

The preferred surfactants are SDS and Tween 20, which may be used in the range from 0.01% to 1% and particularly at 0.1% relative to the total weight of the composition containing them.

### Dyeing Process Using i) to v)

According to a particular embodiment of the invention, the dyeing process comprises the following steps:

the first step consists in preparing composition B as defined previously, in particular in the form of a poultice as defined previously, using indigo-producing plant powder i), into which is optionally incorporated hydrogen peroxide or one or more hydrogen peroxide-generating systems ii) as defined previously and optionally one or more metal salts as defined previously iv) and optionally one or more surfactants v) as defined previously;

in the second step, composition B as defined previously is applied to the keratin fibres and is left on the said materials preferably for a minimum time of 30 minutes, preferentially a time ranging from 30 minutes to 12 hours and better still ranging from 1 hour to 4 hours;

in the third step, the keratin fibres are rinsed with water until the poultice has disappeared, preferably without shampooing;

the keratin fibres may then be either dried or left wet, preferably left wet.

It is understood that between the second and the third steps, a composition C comprising one or more alkaline agents as defined previously and optionally one or more surfactants v) as defined previously is applied to the said fibres, this composition preferably comprising one or more (bi)carbonates as defined previously.

According to another particular embodiment of the invention, the dyeing process is performed in several steps:

the first step consists in preparing composition B of the invention as described previously;

in the second step, composition B is left to stand for 30 minutes to 4 hours and preferably between 30 minutes and 1 hour, and composition B is then applied and left on the said fibres preferably for a minimum time of 30 minutes (preferably ranging from 30 minutes to 24 hours and better still from 1 hour to 12 hours);

in the third step, the keratin fibres are rinsed with water until the poultice has disappeared, preferably without shampooing;

the keratin fibres may then be dried or left to dry naturally, without a hairdryer; preferably, the keratin fibres are left wet.

The aqueous composition A, preferably water, water and an oil, water, an oil and a co-solvent and cosmetic additives, mixed with the indigo-producing plant powder i) used in the first step, may be at room temperature or at a higher temperature, in particular at a temperature ranging from 40° C. to 98° C.

According to another embodiment of the invention, the indigo-producing plant powder i) is mixed with or crumbled into an aqueous composition, preferably water, at a temperature below 40° C., in particular between 10° C. and 40° C.

Preferably, the ratio of the weight amount of indigo-producing plant powder i)/weight amount of aqueous composition A, preferably water, ranges from 1/1 to 1/3 and is preferably 1/2.

According to a particularly advantageous process, after applying the alkaline agent(s) ii) preferably to wet keratin fibres, the said fibres undergo a heat treatment according to a) and b) as defined below or alternatively are after the third step in which the keratin fibres are rinsed with water until composition B has disappeared, preferably without shampooing;
a) either dried by heat with a heat source (convection, conduction or radiation) by passing over, for example, a stream of a warm gas such as air necessary to evaporate off the solvent(s); heat sources that may be mentioned include a hairdryer, hairdrying hoods, a hair-straightening iron, an infrared ray dispenser and other standard heating appliances;
b) or the application of ceramic heating tongs from 80° C. to 220° C. and more preferentially from 120° C. to 180° C.; preferably, step b) is preferred.

Preferably, the temperature of application of composition B ranges from room temperature (15° C. to 25° C.) to 80° C. and more particularly from 15° C. to 45° C.

After applying the poultice according to the invention, the head of hair may advantageously be subjected to a heat treatment by heating at a temperature ranging from 30° C. to 60° C. In practice, this operation may be performed using a hairstyling hood, a hairdryer, an infrared ray dispenser or any other standard heating appliance.

Use may especially be made, both as means for heating and straightening the hair, of a heating iron at a temperature ranging from 60° C. to 230° C. and preferably from 120° C. to 180° C.

As regards heat with a heat source (convection, conduction or radiation) by passing over, for example, a stream of a warm gas such as air necessary to evaporate off the solvent(s), heat sources that may be mentioned include a hairdryer, hairdrying hoods, a hair-straightening iron, an infrared ray dispenser and other standard heating appliances. The application of ceramic heating tongs from 80° C. to 220° C. and more preferentially from 120° C. to 180° C. may advantageously replace the other techniques in so far as it is faster.

A particular mode of the invention relates to a dyeing process which is performed at room temperature (25° C.).

### I) EXAMPLES OF DYEING

The following compositions were prepared:

The percentages are indicated on a weight basis relative to 100 g of composition.

### Compositions B:

| | Compositions | |
|---|---|---|
| | B | |
| | B1 | B2 |
| Powdered leaves of indigo plant (Indigofera tinctoria) i) | 25% | 25% |
| Hydrogen peroxide iii) | | 0.1% |
| water qs 100 | 75% | 74.9% |

The described ingredients i) to v) are dissolved or dispersed in the relative amounts described in the table below in hot water (25° C. to 60° C.) in a bowl. The indigo plant powder is placed in another bowl and the contents of the first bowl are added to the indigo plant powder. The whole is homogenized with a spoon or spatula.

The poultice obtained is very creamy, and is applied at the time of use to the keratin fibres, at a rate of 20 g of poultice per gram of hair, totally impregnating the keratin fibres from the root to the end, at room temperature.

The poultice is applied to dry natural grey hair containing 90% white hairs, with a leave-on time of 60 minutes.

The hair is rinsed thoroughly.

The compositions C are added to wet hair.

### Compositions C:

| Compositions C | Alkaline agents ii) and pH |
|---|---|
| C1 | NaOH/glycine buffer, 0.5M pH 11 |
| C2 | NaOH/glycine buffer, 0.5M pH 10 |
| C3 | NaOH/glycine buffer, 0.5M pH 9 |
| C4 | Sodium carbonate/sodium bicarbonate buffer, |
| | 0.5M pH 11 |
| C5 | Sodium carbonate/sodium bicarbonate buffer, |
| | 0.5M pH 10 |
| C6 | Sodium carbonate/sodium bicarbonate buffer, |
| | 0.5M pH 9 |
| C7 | Tris/HCl buffer, 0.5M, pH 9 |
| C8 | Sodium phosphate buffer, 0.5M, pH 9 |
| C9 | Ammonium bicarbonate, 0.5M, pH 8.5 |
| C10 | Potassium bicarbonate, 0.5M, pH 8.4 |
| C11 | Sodium bicarbonate, 0.5M, pH 8.2 |
| C12 | Potassium carbonate/potassium bicarbonate buffer, |
| | 0.5M pH 9 |

### Colorimetric Results (Dyed Locks)

The colour of the locks was evaluated in the CIE L*a*b* system, using a Minolta CM-2000 spectrocolorimeter.

In this L*a*b* system. L* represents the intensity of the colour, a* indicates the green/red colour axis and b* indicates the blue/yellow colour axis.

The lower the value of L*, the darker or more intense the colour.

Improvement of the Dyeing Kinetics as Regards the Intensity

| | | |
|---|---|---|
| | | ΔL at 22 hours |
| Treatment | L | and 5 days |

Comparative

| | | |
|---|---|---|
| B1; T = 0 (T0) | 37 | DL₂₂ₕ = 2.85 |
| B1; T = 1 hour (T 1h) | 34.7 | |
| B1; T = 5 hours (T 5h) | 34.24 | |
| B1; T = 22 hours (T 22h) | 34.15 | |
| B1; T = 5 days (T 5d) | 29.16 | DL₅ⱼ = 7.84 |

Invention

| | | |
|---|---|---|
| B1 + post-treatment C1; T0 | 28.28 | 0.10 |
| B1 + post-treatment C1; T1h | 28.19 | |
| B1 + post-treatment C1; T5h | 26.96 | |
| B1 + post-treatment C1; T22h | 28.18 | |
| B1 + post-treatment C1; T5d | 27.67 | 0.61 |
| B1 + post-treatment C2; T0 | 31.85 | 0.47 |
| B1 + post-treatment C2; T1h | 32.41 | |
| B1 + post-treatment C2; T5h | 31.22 | |
| B1 + post-treatment C2; T22h | 31.38 | |
| B1 + post-treatment C2; T5d | 31.27 | 0.58 |
| B1 + post-treatment C3; T0 | 31.99 | 1.07 |
| B1 + post-treatment C3; T1h | 33.02 | |
| B1 + post-treatment C3; T5h | 31.91 | |
| B1 + post-treatment C3; T22h | 30.92 | |
| B1 + post-treatment C3; T5d | 30.16 | 1.83 |
| B1 + post-treatment C4; T0 | 28.28 | 0.41 |
| B1 + post-treatment C4; T1h | 28.39 | |
| B1 + post-treatment C4; T5h | 27.51 | |
| B1 + post-treatment C4; T22h | 27.87 | |
| B1 + post-treatment C5; T0 | 29.28 | 1.68 |
| B1 + post-treatment C5; T1h | 29.38 | |
| B1 + post-treatment C5; T5h | 29.24 | |
| B1 + post-treatment C5; T22h | 27.6 | |
| B1 + post-treatment C5; T5d | 27.75 | 1.53 |
| B1 + post-treatment C6; T0 | 30.99 | 3.47 |
| B1 + post-treatment C6; T1h | 28.68 | |
| B1 + post-treatment C6; T5h | 27.33 | |
| B1 + post-treatment C6; T22h | 27.52 | |
| B1 + post-treatment C6; T5d | 27.38 | 3.61 |
| B1 + post-treatment C7; T0 | 30.21 | 2.81 |
| B1 + post-treatment C7; T1h | 29.64 | |
| B1 + post-treatment C7; T5h | 29.86 | |
| B1 + post-treatment C7; T22h | 27.4 | |
| B1 + post-treatment C7; T5d | 26.67 | 3.54 |
| B1 + post-treatment C8; T0 | 33.49 | 3.79 |
| B1 + post-treatment C8; T1h | 31.68 | |
| B1 + post-treatment C8; T5h | 30.6 | |
| B1 + post-treatment C8; T22h | 29.7 | |
| B1 + post-treatment C9; T0 | 30.37 | 1.02 |
| B1 + post-treatment C9; T1h | 30.24 | |
| B1 + post-treatment C9; T5h | 30.25 | |
| B1 + post-treatment C9; T22h | 29.35 | |
| B1 + post-treatment C9; T5d | 29.6 | 0.77 |
| B1 + post-treatment C10; T0 | 32.1 | 3.42 |
| B1 + post-treatment C10; T1h | 30.55 | |
| B1 + post-treatment C10; T5h | 29.14 | |
| B1 + post-treatment C10; T22h | 28.68 | |
| B1 + post-treatment C10; T5d | 29.59 | 2.51 |
| B1 + post-treatment C11; T0 | 31.74 | 1.76 |
| B1 + post-treatment C11; T1h | 30.73 | |
| B1 + post-treatment C11; T5h | 29.36 | |
| B1 + post-treatment C11; T22h | 29.98 | |
| B1 + post-treatment C11; T5d | 30.39 | 1.35 |
| B1 + post-treatment C12; T0 | 30.69 | 2.72 |
| B1 + post-treatment C12; T1h | 30.77 | |
| B1 + post-treatment C12; T5h | 29.83 | |
| B1 + post-treatment C12; T22h | 27.97 | |
| B1 + post-treatment C12; T5d | 28.49 | 2.2 |

The experiments are performed at room temperature.

It is seen that for all the compositions tested of the invention, the alkaline post-treatment makes it possible to obtain an excellent intensity after one hour or even a few hours, whereas, without the alkaline post-treatment step, it takes at least 5 days. Furthermore, all the intensities obtained are significantly better with the process according to the invention than with the comparative process.

The NaOH/glycine pH 10 buffer (composition C2) represents a good compromise between the increase in kinetics and stabilization of the colour.

### Effect of Heat

| Treatment | L | a* | b* | ΔE |
|---|---|---|---|---|
| Comparative | | | | |
| B1 + 30 minute waiting period - T0 | 35.5 | −5.17 | −5.94 | 33.3 |
| B1 + post-treatment in an oven (50° C.) | 35.86 | −6.35 | 0.18 | 30.0 |
| for 30 minutes | | | | |
| B1 + straightening tongs (150° C.) | 29.18 | 2.56 | 1.38 | 34.8 |
| Invention | | | | |
| B1 + C11 + post oven (50° C.) 30 min | 27.84 | −0.16 | −6.31 | 39.3 |
| B1 + C11 + straightening tongs (150° C.) | 28.41 | −0.69 | −4.08 | 37.8 |

It is preferable for the keratin fibres to remain wet before applying heat.

It is seen that the combined action of the alkaline post-treatment with heat according to the invention makes it possible to form the blue colour very rapidly, whether in an oven at 50° C. for 30 minutes or under the action of straightening tongs on wet hair at 150° C., virtually immediately, when compared with the comparative process which does not use an alkaline post-treatment.

Fixing of the Blue Colour of Keratin Fibres Dyed with Indigo and Limitation of the Colour Change Towards Red

The alkaline agents according to the invention and in particular potassium carbonate or sodium carbonate may be used at a pH of less than or equal to 11.

| Treatment | L* | a* | b* |
|---|---|---|---|
| Comparative | | | |
| B1 + T5d | 29.16 | 2.94 | −4.31 |
| Invention | | | |
| B1 + post-treatment C4; T5d | 26.01 | 2.63 | −6.67 |
| B1 + post-treatment C5; T5d | 27.75 | 0.89 | −5.57 |
| B1 + post-treatment C6; T5d | 27.38 | 2.39 | −6.77 |
| B1 + post-treatment C12; T5d | 28.49 | 1.08 | −5.43 |

The results show that the alkaline treatments make it possible to prevent the change towards red over time, while at the same time maintaining excellent colouring power L lower than for the comparative.

Advantageously, the two improvements may be combined:

oxidizing agent of hydrogen peroxide type to improve the kinetics and co-treatment (SDS. Tween or metal salts such as the Fe(II) salts) or alkaline post-treatment to avoid the colour change.

| Treatment | a* | b* |
|---|---|---|
| Comparative | | |
| B2 | 0.35 | −5.72 |
| B2 + 1.5 months | 9.11 | −10.55 |
| Invention | | |
| B2 then post-treated C11 (invention) | 0.4 | −4.68 |
| B2 then post-treated C11 + 1.5 months (invention) | 1.86 | −5.87 |

It is seen that the process of the invention makes it possible to avoid the colour change towards red when compared with the comparative process without alkaline post-treatment.

1.-15. (canceled)

16. A method for dyeing keratin materials, in particular human keratin fibers, comprising:
(a) preparing a composition B, comprising:
an indigo-producing plant powder,
an aqueous composition A,
optionally, hydrogen peroxide or at least one hydrogen peroxide-generating system, and
optionally, at least one metal salt;
(b) applying composition B to the keratin fibers; and
(c) applying a composition C to the keratin fibers, composition C comprising at least one alkaline agent,
wherein composition C has a pH ranging from about 7.5 to about 11.5.

17. The method according to claim 16, wherein the indigo-producing plant is chosen from:
Indigofera, Indigofera tinctoria, Indigo suffruticosa, Indigofera articulata, Indigofera arrecta, Indigofera gerardiana, Indigofera argenta, Indigofera indica, or Indigofera longiracemosa;
Isatis or Isatis tinctoria;
Polygonum, Persicaria, or Polygonum tinctorium (Persicaria tinctoria);
Wrightia or Wrightia tinctoria;
Calanthe or Calanthe veratrifolia; or
Baphicacanthus or Baphicacanthus cusia.

18. The method according to claim 16, wherein the indigo-producing plant powder is indigofera.

19. The method according to claim 16, wherein the indigo-producing plant powder is present in an amount ranging from about 10% to about 99% by weight, relative to the total weight of composition A.

20. The method according to claim 16, wherein the indigo-producing plant powder is present in a ratio of indigo-producing plant powder to composition A ranging from about 1 to 1 to about 1 to 3.

21. The method according to claim 16, wherein composition A or composition B is acidic and has a pH ranging from about 2 to about 6.5.

22. The method according to claim 16, wherein the at least one alkaline agent is chosen from aqueous ammonia; alkali metal or alkaline-earth metal hydroxides buffered with at least one amino acid, glycine, carbonate, bicarbonate, or hydrogen carbonate; carbonates buffered with bicarbonate to obtain a pH less than 11.5; phosphates of alkali metals, alkaline-earth metals, sodium, or potassium; alkanolamines, mono-, di- or triethanolamines; mono-, di- or tri(hydroxymethyl)aminomethanes, amino acids, the compounds of formula (I) below: 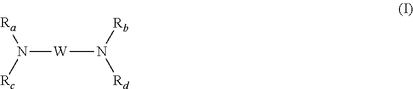
wherein W represents a (C₁-C₆)alkylene group, optionally substituted with a hydroxyl, amino group, or C₁-C₄ alkyl radical; Rₐ, R_{b}, R_{c} and R_{d}, which may be identical or different, are chosen from a hydrogen atom, a C₁-C₄ alkyl, or C₁-C₄ hydroxyalkyl radical;
or derivatives thereof.

23. The method according to claim 16, wherein the at least one alkaline agent is chosen from alkali metal; ammonium carbonates; alkali metal; ammonium bicarbonates; alkali metal carbonates buffered with alkali metal bicarbonates to obtain a pH less than 12.0; hydroxides of alkali metals, alkaline-earth metals, or NaOH buffered with at least one amino acid to obtain a pH<12.0; phosphates of alkali metals, alkaline-earth metals, sodium, potassium, alkanolamines, or tri(hydroxymethyl)aminomethane.

24. The method according to claim 16, comprising hydrogen peroxide.

25. The method according to claim 16, wherein the at least one or alkaline agent is present in composition C in a concentration ranging from 0.1 M to 1 M and wherein composition C has a pH greater than or equal to 7.5.

26. The method according to claim 16, wherein the at least one metal salt comprises a transition metal, rare-earth metal, manganese, iron, zinc, titanium, zirconium, molybdenum, tungsten, vanadium, or a metal of oxidation state I or II.

27. The method according to claim 26, wherein the at least one metal salt comprises a metal of oxidation state I or II.

28. The method according to claim 16, wherein the at least one metal salt is organic, and complexed with two carboxylate groups, such as those corresponding to formula (III) below:
R—C(O)—O-M-O—C(O)—R′ (III)
and the solvates and enantiomers thereof, wherein:
M is chosen from a metal (II) or metal²⁺ in oxidation state 2, and
R and R′, which may be identical or different, represent a (C₁-C₆)(poly)hydroxyalkyl group.

29. The method according to claim 16, wherein the at least one metal salt is chosen from citrates, lactates, glycolates, gluconates, acetates, propionates, fumarates, oxalates, or tartrates.

30. The method according to claim 16, further comprising at least one surfactant, preferably nonionic or anionic surfactants; particularly as a post-treatment, i.e. the surfactant(s) v) are applied after ingredient i), and they are preferably with ingredient ii), in the composition C as defined in any one of claims 16, 21 and 22.

31. The method according to claim 30, wherein the at least one surfactant is applied to the keratin fibers after composition B.

32. The method according to claim 30, wherein the at least one surfactant is present in composition C.

33. The method according to claim 30, wherein the at least one surfactant is chosen from:
optionally oxyalkylenated C₆-C₃₀ fatty acid esters of sorbitan such as those corresponding to formula (IV) below: 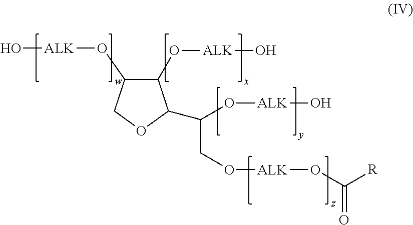
and the optical isomers or hydrates thereof,
wherein:
ALK, which may be identical or different, is chosen from a linear or branched (C₁-C₆)alkylene group;
x, y and z, which may be identical or different, are an integer ranging from 0 to 100, wherein the sum of x+y+z is an integer ranging from 1 to 100;
R is chosen from a linear or branched (C₆-C₃₀)alkyl;
further wherein, the sorbitan esters are oxyethylenated with a number of moles of ethylene oxide ranging from 15 to 30; or
surfactants containing carboxylate, sulfate, sulfonate, sulfoacetate, sulfosuccinate, phosphate, isethionate, sarcosinate, glutamate, lactylate, taurate, fatty acid salt, galactosideuronic salt, carboxylic ether acid salt anionic groups, or mixtures thereof.

34. The method according to claim 16, further comprising a heat treatment after the application of composition C or after rinsing the keratin fibers after the application of composition B, wherein the heat treatment is chosen from:
a) drying the keratin fibers by heat with a heat source (convection, conduction or radiation) by passing over, for example, a stream of a warm gas such as air necessary to evaporate off the solvent(s); or
b) applying to the keratin fibers ceramic heating tongs with a temperature ranging from 80° C. to 220° C.
